(19) United States
(12) Patent Application Publication
(10) Pub. No.: US 2019/0295701 A1
(43) Pub. Date: Sep. 26, 2019

(54) DRUG ADMINISTRATION TIMING

(71) Applicants: Ritankar Das, Hayward, CA (US); Samson Mataraso, Hayward, CA (US); Jacob Calvert, Hayward, CA (US)

(72) Inventors: Ritankar Das, Hayward, CA (US); Samson Mataraso, Hayward, CA (US); Jacob Calvert, Hayward, CA (US)

(21) Appl. No.: 15/934,985

(22) Filed: Mar. 24, 2018

(51) Int. Cl. G16H 20/10; G16H 50/70; G16H 10/60

(52) U.S. Cl. CPC G16H 20/10; G16H 10/60; G16H 50/70

(57) ABSTRACT

Methods for determining the time at which a drug should be administered, based on patient electronic health record data. Machine learning techniques are used to correlate trends in health record data with successful drug treatment, ultimately anticipating the optimal time of drug administration. Multiple types of data, including demographic, physiological, treatment, and clinical notes data, can be used to train the classification component. Multiple patient populations can be used as sources of patient data for training classification component. Data input requirements, dimensionality, and performance metrics may be optimized.

### FIELD OF THE INVENTION

The present disclosure generally relates to patient monitoring and diagnosis, and in particular to the prediction of optimal drug administration time for use in clinical trials.

### BACKGROUND OF THE INVENTION

Clinicians have traditionally made decisions regarding which medication to prescribe for a patient's health condition or illness based on medication information that they have memorized or can quickly look up in a reference guide. The clinician may have learned about a given kind of medication, for example, a specific anti-inflammatory agent, from a medical journal, advertisement, educational lecture, or other means. Generally, drugs target specific physiologic pathways through their mechanism of action, and a disease which presents very similarly may be caused by different physiologic pathways. In certain heterogeneous disease populations, for example, sepsis, although there are many others, drug effectiveness and toxicity depend on the timing of administration. The optimal drug administration time reflects the timing of the patient's underlying disease progression. As disease progression differs from case-to-case and patient-to-patient, there is need for a tool which can identify the time of administration for which effectiveness is maximal and toxicity is minimal. There currently exists limited ways to do this. Therefore, there exists a need for a method of deciding when to effectively administer a drug based on whether or not the mechanism of action of the drug will treat that specific manifestation of the condition.

### SUMMARY OF THE INVENTION

The presently disclosed embodiments are directed to solving one or more of the problems presented in the prior art, described above, as well as providing additional features that will become readily apparent by reference to the following detailed description when taken in conjunction with the accompanying drawings.

In an embodiment, the disclosure provides a method of administering a drug treatment to a candidate patient, which includes: acquiring patient data from a plurality of patients; comparing the acquired patient data to classified anonymized patient health record data; and administering the drug treatment to the candidate patient based on whether or not the timing of the drug treatment is efficacious in treating a specific manifestation of the candidate patient's condition as evidenced by the anonymized patient health record data.

In another embodiment, the disclosure provides a method of administering a drug treatment to a candidate patient, wherein the anonymized patient health record data includes (i) patient demographics, (ii) measurements of vital signs, (iii) physiological monitor data, (iv) the ward in which the patient is staying, (v) diagnosis and treatment information, (vi) lab test results, (vii) medication data, (viii) patient outcome information, (ix) clinical notes, and/or (x) patient medical history.

In another embodiment, the disclosure provides a method of administering a drug treatment to a candidate patient, wherein the anonymized patient health record data reflects the nature of the patient population served by the hospital or clinic in terms of patient demographics, rates of disease incidence, and/or treatment practices.

In another embodiment, the disclosure provides a method of administering a drug treatment to a candidate patient, wherein the anonymized patient health record data is sourced from a database of the plurality of patients, a database of one or more care centers and patient populations, or from a database of multiple care centers and patient populations.

In another embodiment, the disclosure provides a method of administering a drug treatment to a candidate patient, wherein the anonymized patient health record data is collected at a standard interval.

In another embodiment, the disclosure provides a method of administering a drug treatment to a candidate patient, wherein the anonymized patient health record data includes at least one patient labeled positively for a gold standard which indicates a patient as reaching a certain point in a disease pathway when the drug is expected to be effective.

In another embodiment, the disclosure provides a method of identifying a candidate patient for drug treatment, wherein the labeled patient data includes a positive label for a gold standard which represents a specific progression through the disease pathway.

In another embodiment, the disclosure provides a method of administering a drug treatment to a candidate patient, wherein the anonymized patient health record data continually improve as new data becomes available.

In another embodiment, the disclosure provides a method of administering a drug treatment to a candidate patient, wherein the classified anonymized patient health record data includes an operating point that balances measurements of specificity and sensitivity in order to effectively treat as many patients as possible.

In another embodiment, the disclosure provides a method of administering a drug treatment to a candidate patient, wherein appropriate timing of the administration of the drug treatment reduces drug toxicity and/or increases efficacy.

In another embodiment, the disclosure provides a method of administering a drug treatment to a candidate patient, wherein the drug treatment is administration of resatorvid, eritoran, CytoFab, trigriluzole, or a 5-HT4 agonist or any salt, acid, base, hydrate, solvate, ester, isomer, polymorph, metabolite or prodrug thereof.

In another embodiment, the disclosure provides a method of using a machine learning algorithm for administering a drug treatment to a candidate patient, which includes acquiring anonymized patient health record data from a plurality of patients; comparing acquired patient data to the acquired anonymized patient health record data; and administering the drug treatment to the candidate patient based on whether or not the timing of the drug treatment is efficacious in treating a specific manifestation of the candidate patient's condition as evidenced by the anonymized patient health record data.

In another embodiment, the disclosure provides a method of using a machine learning algorithm for administering a drug treatment to a candidate patient, wherein the anonymized patient health record data includes (i) patient demographics, (ii) measurements of vital signs, (iii) physiological monitor data, (iv) the ward in which the patient is staying, (v) diagnosis and treatment information, (vi) lab test results, (vii) medication data, (viii) patient outcome information, (ix) clinical notes, and/or (x) patient medical history.

In another embodiment, the disclosure provides a method of using a machine learning algorithm for administering a drug treatment to a candidate patient, wherein the anonymized patient health record data reflects the nature of the patient population served by the hospital or clinic in terms of patient demographics, rates of disease incidence, and/or treatment practices.

In another embodiment, the disclosure provides a method of using a machine learning algorithm for administering a drug treatment to a candidate patient, wherein the anonymized patient health record data is sourced from a database of the plurality of patients, a database of one or more care centers and patient populations, or from a database of multiple care centers and patient populations.

In another embodiment, the disclosure provides a method of using a machine learning algorithm for administering a drug treatment to a candidate patient, wherein the anonymized patient health record data is collected at a standard interval.

In another embodiment, the disclosure provides a method of using a machine learning algorithm for administering a drug treatment to a candidate patient, wherein the anonymized patient health record data includes at least one gold standard patient data that identifies that patient is progressing through a disease pathway for which the drug is expected to be effective.

In another embodiment, the disclosure provides a method of using a machine learning algorithm for administering a drug treatment to a candidate patient, wherein the at least one gold standard patient data includes a specific progression through the disease pathway.

In another embodiment, the disclosure provides a method of using a machine learning algorithm for administering a drug treatment to a candidate patient, wherein the anonymized patient health record data continually improve as new data becomes available.

In another embodiment, the disclosure provides a method of using a machine learning algorithm for administering a drug treatment to a candidate patient, wherein the classified anonymized patient health record data includes an operating point that balances measurements of specificity and sensitivity in order to effectively treat as many patients as possible.

In another embodiment, the disclosure provides a method of using a machine learning algorithm for administering a drug treatment to a candidate patient, wherein appropriate timing of the administration of the drug treatment reduces drug toxicity.

In another embodiment, the disclosure provides a method of using a machine learning algorithm for administering a drug treatment to a candidate patient, wherein the drug treatment is administration of resatorvid, eritoran, CytoFab, trigriluzole, or a 5-HT4 agonist or any salt, acid, base, hydrate, solvate, ester, isomer, polymorph, metabolite or prodrug thereof.

### BRIEF DESCRIPTION OF THE DRAWINGS

The present disclosure, in accordance with one or more various embodiments, is described in detail with reference to the following figures. The drawings are provided for purposes of illustration only and merely depict exemplary embodiments of the disclosure. These drawings are provided to facilitate the reader's understanding of the disclosure and should not be considered limiting of the breadth, scope, size, or applicability of the disclosure. It should be noted that for clarity and ease of illustration these drawings are not necessarily made to scale.

FIG. 1 illustrates an embodiment of the different types of data that can be used that are relevant to the classification or prediction task;

FIG. 2 illustrates an embodiment of the possibilities for training on different populations of data;

FIG. 3 illustrates an embodiment of a gold standard determination of positive-class and negative-class labels;

FIG. 4 illustrates an embodiment of decision-making processes;

FIG. 5 illustrates an embodiment of a timeline of disease progression; and

FIG. 6 illustrates an embodiment of a timeline of patients being excluded or allocated to control and experimental groups on the basis of their classification.

### DETAILED DESCRIPTION OF EXEMPLARY EMBODIMENTS

The following description is presented to enable a person of ordinary skill in the art to make and use embodiments described herein. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the disclosure. The word “exemplary” is used herein to mean “serving as an example illustration.” Any aspect or design described herein as “exemplary” is not necessarily to be construed as preferred or advantageous over other aspects or designs. Thus, the present disclosure is not intended to be limited to the examples described herein and shown but is to be accorded the scope consistent with the claims.

As used herein, reference to any biological drug includes any fragment, modification or variant of the biologic, including any pegylated form, glycosylated form, lipidated form, cyclized form or conjugated form of the biologic or such fragment, modification or variant or prodrug of any of the foregoing. As used herein, reference to any small molecule drug includes any salt, acid, base, hydrate, solvate, ester, isomer, or polymorph thereof or metabolite or prodrug of any of the foregoing.

It should be understood that the specific order or hierarchy of steps in the process disclosed herein is an example of exemplary approaches. Based upon design preferences, it is understood that the specific order or hierarchy of steps in the processes may be rearranged while remaining within the scope of the present disclosure. Any accompanying method claims present elements of the various steps in a sample order and are not meant to be limited to the specific order or hierarchy presented.

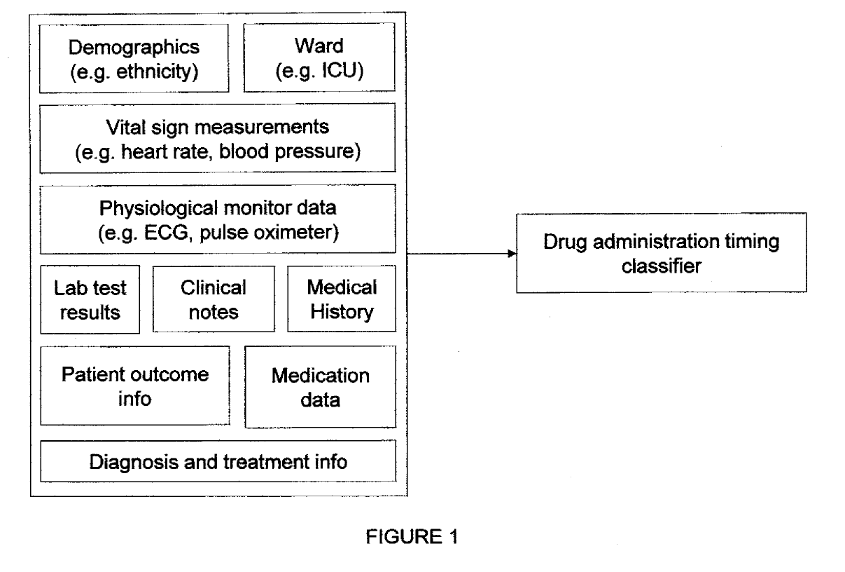

FIG. 1 illustrates an embodiment of the different types of data that can be used that are relevant to the classification or prediction task. The use of machine learning techniques necessitates the availability of data relevant to the classification or prediction task, on which to train the machine learning algorithm. In the context of the current disclosure, these data are typically anonymized patient health records, which can include amongst other information (i) patient demographics, (ii) measurements of vital signs, (iii) physiological monitor data, (iv) the ward in which the patient is staying, (v) diagnosis and treatment information, (vi) lab test results, (vii) medication data, (viii) patient outcome information, (ix) clinical notes, and (x) patient medical history. Any or all of these types of data, along with other types of patient health information, can serve as inputs to the training procedure associated with a machine learning algorithm.

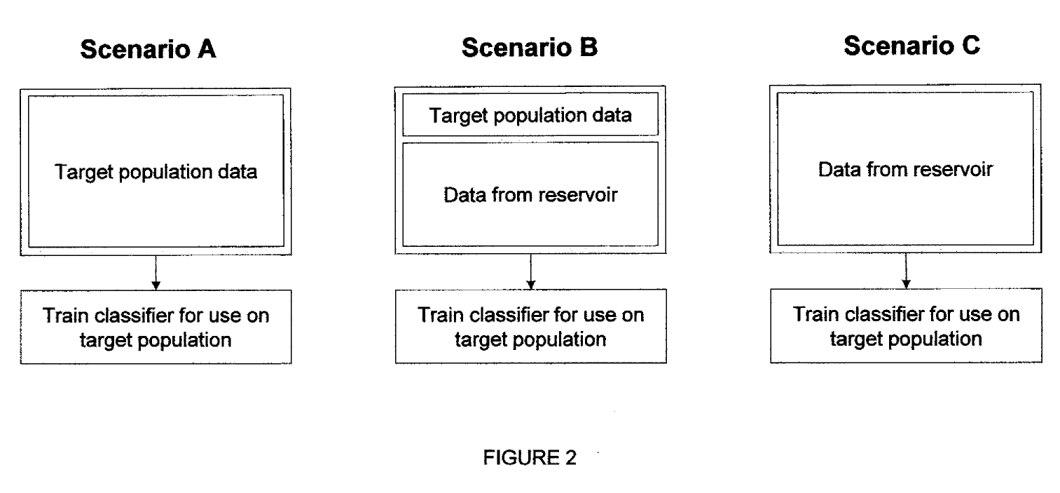

FIG. 2 illustrates an embodiment of the possibilities for training on different populations of data. Patient health record information can be collected and stored by the hospitals or clinics which deliver patient care. In this case, the health record data may reflect the nature of the patient population served by the hospital or clinic, in terms of patient demographics, rates of disease incidence, treatment practices, and other information potentially relevant to a prediction or classification task. Accordingly, when using a machine learning algorithm to develop a prediction or classification tool for use at a hospital or clinic, it may be useful to train with data from the relevant patient population or data from a similar population.

Patient health record data for training a machine learning algorithm can be sourced from a pre-existing or reservoir of data, consisting of anonymized health records from one or more care centers and patient populations, typically with some variability in the types and amount of data that are available for patients in the data set. Alternatively, health record data can be obtained from multiple care centers and patient populations. For example, the Medical Information Mart for Intensive Care III (MIMIC-III) is a publicly-accessible database of anonymized patient health record information, collected from Beth Israel Deaconess Medical Center (Boston, Mass.) between 2001 and 2012, which contains many of the aforementioned types of health data for tens of thousands of patients. A database like MIMIC-III would contrast with patient health record data available from the Veterans Health Administration, for example, which consists of many more health centers, spread across many states and cities. Accordingly, the types and resolution of health data available from such a data set would likely vary more.

When training a machine learning algorithm, it is typically ideal to train on a data set collected from the same population on which the resulting tool is intended to be applied. If there are sufficient training data from the target care center or population, the training procedure can proceed without modification as specified by the machine learning algorithm. If, however, there are not sufficient available data, the training procedure may be modified to rely on both a reservoir of health record data, as well as a small collection of clinic- or population-specific data; alternatively, the training procedure may rely entirely on a reservoir of data. A typical way to modify the training procedure in the former case is with the techniques of transfer learning, wherein the machine learning algorithm is first trained on a reservoir of data, before being trained further on the target dataset in such a way as to emphasize the examples it contains.

Ideally, all measurements relevant to a prediction or classification task are measured frequently, at a standard interval, e.g. one measurement every hour. However, patient health record data include types of data with varying frequencies of measurement and, as such, it is often convenient to standardize the frequencies with which new measurements are assessed by the prediction or classification tool resulting from the training procedure. For example, to produce a new patient classification every hour, the time series of measurements may be partitioned or “binned” into one-hour increments and relayed to the classifier accordingly.

As there will likely be bins during which no new measurement is available for a particular patient and type of data, it is standard to implement a data imputation scheme, whereby available data are used to fill-in missing data. The simplest such imputation method is a “carry-forward” rule, where the most recent measurement for a particular input, e.g. a heart rate measurement, can be used in subsequent empty bins. There are other, more complicated methods for data imputation, including the filling of empty bins with the running average of the measurements of the relevant input, or inferring the missing value from a patient with a quantitatively similar trajectory of measurements.

It is also the case that sometimes multiple measurements of the same clinical variable are available within the same binning period. In this case, the frequency of measurements can be standardized by replacing the multiple measurements with the average of their values.

Supervised machine learning algorithms require labeled training data to identify the patterns in the data from which labels can be inferred. For example, to train a classifier for a sepsis from patient health record data, each patient must be assigned a positive or negative label, respectively indicating whether the patient did or did not have sepsis. Typically, before using unlabeled patient data with a supervised learning algorithm, the relevant label is assigned to the patient unambiguously in terms of the data that are available for that patient. This unambiguous way of assigning a label is called a gold standard.

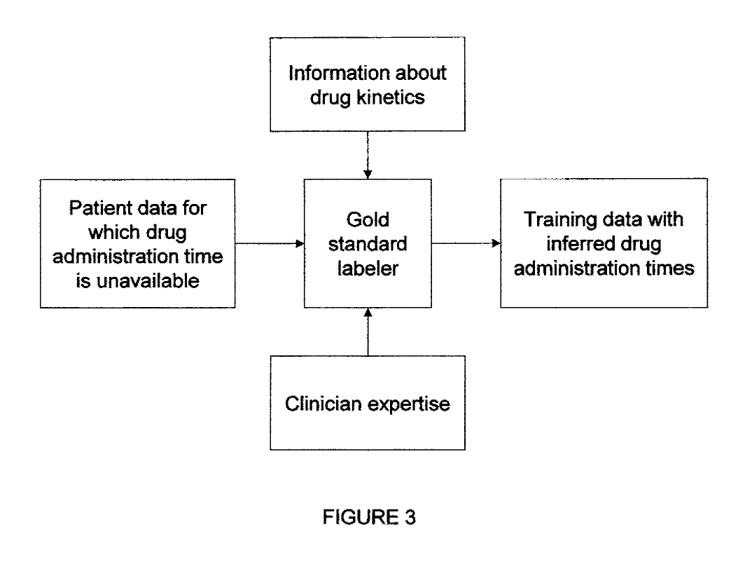

FIG. 3 illustrates an embodiment of a gold standard determination of positive-class and negative-class labels. In many cases, there is no universally agreed-upon gold standard for a label of medical relevance. Accordingly, a choice of gold standard may rely on a combination of standards of medical practice, correlative analyses, data availability constraints, and clinician expertise.

In the context of drug administration timing, a gold standard may or may not be necessary to prepare a data set for training. Consider, for example, a data set for which the time of drug administration is specified, either directly in a field of the electronic health record or in plain language in the clinician's notes. In this case, the training set needs no additional labeling. The drug administration times can be gathered and used in the training procedure.

A more complicated scenario may involve unlabeled training data for which drug administration times were unavailable. In this case, the development of a gold standard would be necessary, and may incorporate detailed information regarding the drug's mechanism of action and kinetics, as well as input from clinician experts. This information could be used to determine the type of response expected in a patient's physiology, as reflected in their vital signs and lab test results, for example. Additionally, such information could be used to determine the latency between drug administration and the appearance of the drug's effects, from which the drug administration time could be inferred. Inferring this latency could incorporate knowledge of a patient's age, weight, and dietary factors, which may affect drug metabolism.

Another relevant training scenario involves a partially completed clinical trial, halted due to concerns of toxicity or other dangers to patient health. In this case, it is known to which patients the drug was administered and when. These labeled data would not require the development of a gold standard before training.

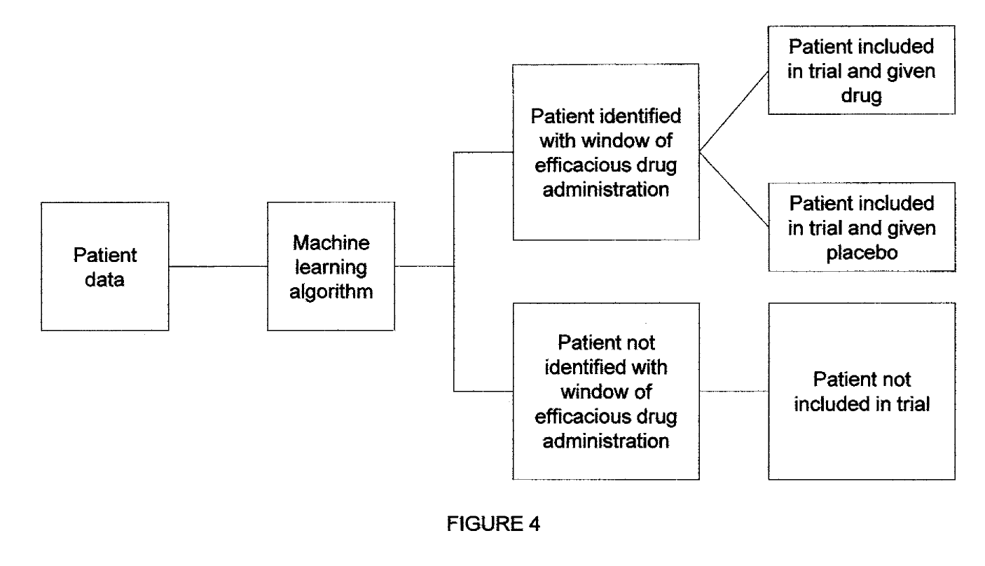

FIG. 4 illustrates an embodiment of a decision-making process, which includes algorithm-based prediction of which patients are in or will soon be in a window of efficacious drug administration. In the context of a trial, patients identified with a period of efficacious drug administration could subsequently be placed uniformly-at-random into experimental and control groups.

The machine learning procedure identifies which features of the data set are most important for the classification or prediction task under consideration. Typically, in the context of the current disclosure, the features are the clinical variables, e.g. vital signs, lab test results, as well as their correlations, e.g. correlation between heart rate and blood pressure, and trends over time, e.g. differences in measurements taken at the beginning and end of a time window. However, it may also be the case that the features consist of all the data points of a patient's stay or, contrastingly, exclusively derivatives thereof.

Consider the example of human recombinant glutamic acid decarboxylase ((GAD, rhGAD65 (Diamyd) or any fragment, modification or variant of rhGAD65, including any pegylated form, glycosylated form, lipidated form, cyclized form or conjugated form of rhGAD65 or such fragment, modification or variant or prodrug of any of the foregoing), which aims to introduce a competing immune response to preserve insulin function in patients with type 1 diabetes. GAD is only effective if there is currently insulin function in a patient. By using an algorithm to determine which patients are in the beginning phases of losing the ability to develop insulin (i.e. responders), GAD can be administered at the correct time to the correct patients, thus having a stronger efficacy signal. Thus, the disclosed algorithms can determine the stage of loss of insulin function in type 1 diabetes patients; glutamic acid decarboxylase (GAD) only is effective in patients with existing insulin function. By detecting patients earlier in stages of insulin function loss, GAD can be given earlier and may be more effective.

Also consider, for example, the TLR-4 inhibitor class of drugs, such as resatorvid (TAK-242, ethyl (6R)-6-[(2-chloro-4-fluorophenyl)sulfamoyl]cyclohexene-1-carboxylate), Takeda Pharmaceutical Company, Ltd.) or eritoran (([(2R,3R,4R,5S,6R)-4-Decoxy-5-hydroxy-6-[[(2R,3R,4R,5S,6R)-4-[(3R)-3-methoxydecoxy]-6-(methoxymethyl)-3-[[(Z)-octadec-11-enoyl]-amino]-5-phosphonatooxyoxan-2-yloxymethyl]-3-(3-oxotetradecanoylamino)oxan-2-yl] phosphoric acid), Eisai Inc.). These drugs inhibit TLR-4 receptors, which are stimulated by pro-inflammatory cytokines. When TLR-4 receptors are stimulated, they release more cytokines, which then stimulate more TLR-4 receptors and so forth, causing a positive feedback loop. This loop is part of the body's natural response to fighting infection; however, if it becomes dysregulated the positive feedback loop can snowball out of control, releasing too many cytokines, known as a cytokine storm. TLR-4 inhibitors aim to suppress these cytokine storms. However, the drug must be given late enough so the body can still have a natural immune response but early enough to suppress a cytokine storm before the positive feedback loop gets out of control. As such, administering this drug at the appropriate time, for example by utilizing the disclosed algorithm, is of utmost importance for it to be efficacious. Thus, the disclosed algorithms can determine patients in the beginning stages of cytokine storms to TLR-4 inhibitors can be given at a more effective time.

If too many features are used in the learning procedure, training can be slow and may overfit the data. Overfitting leads to the appearance of good prediction performance, when tested with the data set on which it was trained, but results in poor generalization to other data sets, i.e. other patient populations. One way to prevent overfitting is by reducing the dimensionality, or number of features, included in the training procedure. Preliminary training and testing can identify those features which are most important to the prediction process; and less important features can then be removed from the training procedure.

The result of the training procedure is, in one form or another, a weighting of the features which can then be used to make predictions on new examples, subject to the features first being constructed from the new data. For classifying aspects of patient health, weighting the various features often leads to a numerical score which reflects the extent to which a given patient is believed to belong to a particular class. By placing a threshold on the score, e.g. patients whose scores are above 10 are determined to have sepsis; and those with scores below 10 do not.

These machine learning algorithms can utilize information about a patient's current medical state and contextualize measurement information. The algorithm contextualizes by looking at deviation from a prior normal. Although there are medically accepted reference ranges for normal values of certain measurements, by analyzing prior measurements the algorithms determine what is normal for a specific patient. This is particularly important in the context of the present disclosure, as successful drug administration timing requires an implicit understanding of a patient's underlying disease progression, the dynamics of which are unique to each patient.

Training a machine learning algorithm must be done cognizantly, as the performance of the algorithm depends heavily on (i) the data used to train the algorithm and (ii) the technique chosen. The data used to train the algorithm must labeled with a gold standard as described in the prior sections. Further, previously described various imputation and feature selection methods must be used to ensure the data is as well formed as possible. Well-formed data allow training processes to identify the best features for predicting patient response to various drugs.

When developing the disclosed companion algorithms multiple learning techniques were utilized and the ones that produce the best area under the receiver operating characteristic curve were identified. Simple techniques such as linear regression was tested, which attempts to find the best equation for a linear regression to fit to the data. Also, more complicated techniques were tested, such as gradient boosted trees. Gradient boosted trees utilize multiple weak prediction models, in this case, decision trees. Decision trees are rule-based models which assign what is in effect a score based on an established set of rules. When combining many decision trees through gradient boosting, very robust predictions are often seen.

As the disclosed algorithms make suggestions about which patient should be receiving treatment based on whether or not a drug's mechanism of action would be efficacious in treating the specific manifestation of their condition at a given time, and as patients are treated accordingly, the patient's health typically improves; however, it may not. In order to reduce false positives, which could cause alarm fatigue, there are inevitably patients that are not treated which should be. Likewise, in order to treat as many patients as possible, the disclosed algorithms sometimes suggests treating patients for whom and at a time when the drug is ineffective. This balance is achieved by selecting an appropriate operating point, which balances these two measurements: specificity and sensitivity.

As the disclosed algorithms run and patients are treated, more data is generated, Another training technique utilized is called online learning. Online learning allows algorithms to continually improve themselves as new data become available. In this context, the disclosed algorithms can learn from their own mistakes. If it suggests prescribing a drug to a patient that ultimately does not have the appropriate physiology to respond to the drug's mechanism of action, that patient will become part of the training data and improve the algorithm's future predictions.

There are many settings for selecting an operating point as indicated as points along an ROC curve. The selection of an operating point is at the discretion of the user. It is a trade-off of sensitivity and specificity; this connects with the number of alerts a user would like to have over a period of time.

In clinical settings, the disclosed algorithms can be implemented directly within an EHR. This direct implementation allows for algorithms to process data in real time from patients as it is entered into their medical records. Further, alerts will be able to be displayed directly to clinicians in a patient's chart. However, external alerts, such as phone calls, are also possible through integration with automated calling APIs. When the disclosed algorithms detect that a patient, who is a candidate for treatment with a given drug, is displaying physiological signals consistent with successful drug administration at a particular time, the relevant clinicians are automatically alerted, through a phone or pager, for example. Clinicians may then administer the drug, based on the algorithm's determination.

Consider for example the sepsis drug CytoFab (Astra Zeneca). In clinical trials CytoFab did reduce plasma tumor necrosis factor alpha concentration in patients; however, this did not result in clinical benefit. Sepsis is a disease which progresses quickly, and as such, the timing of sepsis drug's administration relative to the disease's progression can have a large impact. It is possible that the reduction of tumor necrosis factor alpha concentrations could have an effect on the progression of sepsis if this reduction occurred earlier or later in disease progression. By using training data from patients with varying levels of tumor necrosis factor alpha levels, the disclosed algorithm could be trained to optimize the time of administration of CytoFab, potentially resulting in some clinical benefit. Thus, the disclosed algorithms can determine stage of sepsis progression, allowing drugs like CytoFab to be given at a point when they are most effective.

Another example is the spinocerebellar ataxia drug trigriluzole (Biohaven Pharmaceutical Holding Company). Trigriluzole failed to show efficacy in a clinical trial; however, spinocerebellar ataxia progresses variably across patients. As such, it may be valuable to determine the degree to which the disease has progressed in a patient to determine dosage and when the drug will be efficacious in the first place. The disclosed algorithm can be trained to detect complex and subtle physiologic signals which act as a proxy for the progression of the disease. This information can be used to administer the right amount of trigriluzole at the right time in the disease's progression, potentially improving outcomes. Thus, the disclosed algorithms can determine the stage of progression of spinocerebellar ataxia, allowing trigriluzole to be administered only to patients for whom it would be effective.

Yet another example is the 5-HT4 agonist class of drugs for the treatment of enteral feeding intolerance (EFI). As EFI progresses, it can be more difficult to treat. Therefore, it is valuable to train the disclosed algorithm to detect early signs of EFI so a 5-TH4 agonist can be administered earlier in the disease's progression, likely leading to increased efficacy. Thus, the disclosed algorithms can detect enteral feeding intolerance earlier than existing methods, allowing treatment to be given earlier when it is more effective.

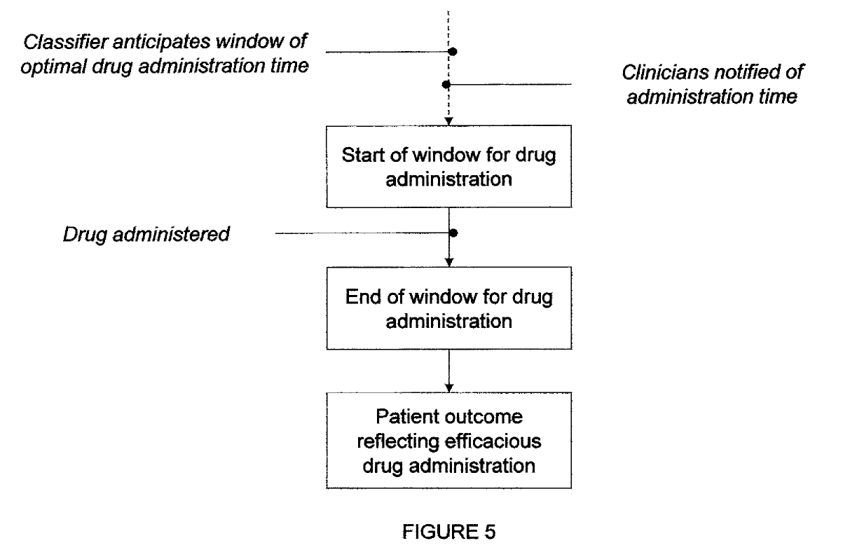

FIG. 5 illustrates an embodiment of a timeline of disease progression. The disclosed algorithms can be used to improve clinical trials. The effectiveness and toxicity of drugs depends on the timing of their administration, which may be difficult to determine consistently in a heterogeneous population of patients. Consequently, clinical trials for promising drugs may be halted or fail. However, by optimizing timing of administration in an automated, consistent manner, clinical drug trials can improve their chance of succeeding. In a clinical trial, such an algorithm could be bundled with the drug as a combined algorithm-drug treatment.

Increasingly, clinical trials utilize adaptive trial designs. Adaptive trial designs begin by looking at a drug's effect in a smaller subgroup of patients to determine likely effect size. Then, as needed, the trial is expanded to more patients to show statistical significance. A smaller initial trial size allows trial coordinators to terminate the trial if initial results show either futility or efficacy. The algorithm described in this disclosure can be used to improve adaptive trial design and reduce the associated costs. In particular, the ability to apply the drug under study at the most advantageous time results in a stronger signal of effectiveness, meaning fewer patients need to be enrolled in order to show statistical significance. Moreover, appropriate timing of administration reduces drug toxicity, meaning less overall risk of the trial ending prematurely. As such, trial size and length can decrease, resulting in substantial cost reduction.

From one perspective, such an algorithm is a method leading to more optimally conducted clinical trials and, from another perspective, it is a tool which naturally augments drugs and can appear on a drug label. This is not unlike the role of companion diagnostic traditionally played by biomarkers or genetic tests. However, implementation as an algorithm improves upon the strengths of these methods, and accounts for their weaknesses, in multiple ways. Biomarkers and genetic tests require an active order from a clinician, which can not only take time but also may be overlooked. An algorithmic companion diagnostic is passive, and always monitoring patients, which means as soon as they display physiological signals indicating the appropriateness of drug administration, they can be treated.

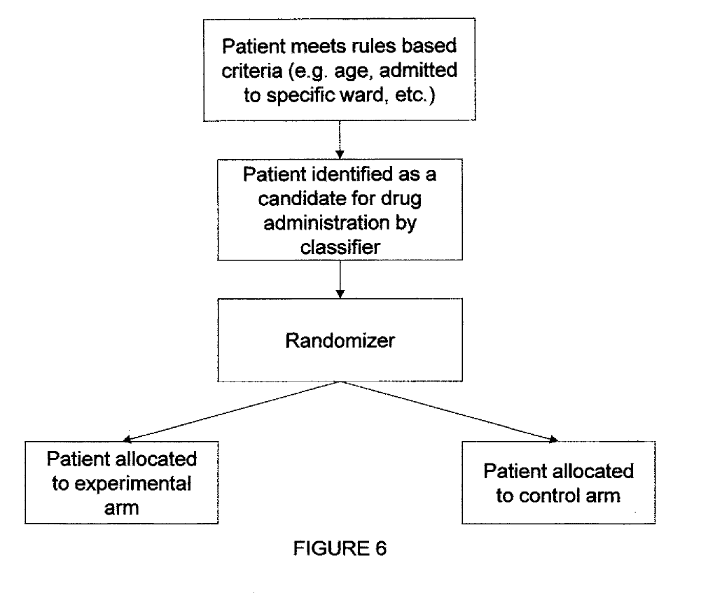

FIG. 6 illustrates an embodiment of an inclusion chart of a clinical trial run with a drug administration timing classifier. Companion algorithms for improving the timing of drug administration benefit from the stores of patient health record data already collected at thousands of hospitals across the US. This means that, unlike biomarkers and genetic tests, there are no expensive R&D procedures related to development. Further, they can be validated on partitioned data—no in vitro tests are required. Lastly, the ability to analyze features which represent more complicated bodily functions, e.g. signals composed of multiple vital signs and lab tests, leads to these algorithms typically having more discriminatory power. Because the algorithm contributes to the performance of a drug, it would become part of the drug's indication upon regulatory approval.

After implementing a classifier resulting from the training procedure, it may be desirable to update the classifier to reflect different priorities of use or to reflect new patient data that have become available for training. Retraining can be completed in batches, that is, by performing the training procedure on an updated training set and choosing an operating point to reflect the use priorities, i.e. picking the sensitivity and specificity of alerts clinicians can expect to receive, which determines the number of alerts clinicians can expect to receive) in the same way as was originally done. Retraining can also be completed continuously as new data become available using an online machine learning technique. Such a method may be relevant in the case of an ongoing trial from which new pairs of drug administration and patient outcome may be derived.

The ability to identify a specific physiologic pathway has broad implications. As described above, an algorithm with discriminatory ability to detect a mechanism of action can increase efficacy signal in a drug for a specific indication. However, the algorithm can also be applied to new indications. Often, drugs work for a specific disease indication because a physiologic pathway is commonly associated with the disease. Utilizing the power of being able to detect a certain physiologic pathway means that a drug's indication can be expanded to other diseases, including those where such a pathway is less common. While traditionally a drug would not be able to show efficacy in a heterogenous disease that can manifest through different physiologic insults, selecting patient responders based on those exhibiting signs of a certain insult could allow efficacy to be reached. Further, it may be possible to have a label based solely upon a disease mechanism, unrelated to any known conditions. This would allow a drug to be used to treat rare or previously undiagnosed conditions, so long as a patient is likely to be helped by the drug.

While the inventive features have been particularly shown and described with reference to preferred embodiments thereof, it will be understood by those in the art that the foregoing and other changes may be made therein without departing from the sprit and the scope of the disclosure. Likewise, the various diagrams may depict an example architectural or other configuration for the disclosure, which is done to aid in understanding the features and functionality that can be included in the disclosure. The disclosure is not restricted to the illustrated example architectures or configurations but can be implemented using a variety of alternative architectures and configurations. Additionally, although the disclosure is described above in terms of various exemplary embodiments and implementations, it should be understood that the various features and functionality described in one or more of the individual embodiments are not limited in their applicability to the particular embodiment with which they are described. They instead can be applied alone or in some combination, to one or more of the other embodiments of the disclosure, whether or not such embodiments are described, and whether or not such features are presented as being a part of a described embodiment. Thus, the breadth and scope of the present disclosure should not be limited by any of the above-described exemplary embodiments.

What is claimed is:

1. A method of administering a drug treatment to a candidate patient, comprising:
acquiring patient data from a plurality of patients;
comparing the acquired patient data to classified anonymized patient health record data; and
administering the drug treatment to the candidate patient based on whether or not the timing of the drug treatment is efficacious in treating a specific manifestation of the candidate patient's condition as evidenced by the anonymized patient health record data.

2. The method of claim 1, wherein the anonymized patient health record data includes (i) patient demographics, (ii) measurements of vital signs, (iii) physiological monitor data, (iv) the ward in which the patient is staying, (v) diagnosis and treatment information, (vi) lab test results, (vii) medication data, (viii) patient outcome information, (ix) clinical notes, and/or (x) patient medical history.

3. The method of claim 1, wherein the anonymized patient health record data reflects the nature of the patient population served by the hospital or clinic in terms of patient demographics, rates of disease incidence, and/or treatment practices.

4. The method of claim 1, wherein the anonymized patient health record data is sourced from a database of the plurality of patients, a database of one or more care centers and patient populations, or from a database of multiple care centers and patient populations.

5. The method of claim 1, wherein the anonymized patient health record data is collected at a standard interval.

6. The method of claim 1, wherein the anonymized patient health record data includes at least one patient labeled positively for a gold standard which indicates a patient as reaching a certain point in a disease pathway when the drug is expected to be effective.

7. The method of claim 6, wherein the data contain at least one patient labeled positively with respect to a designated gold standard which specifies the patient as progressing through a specific disease pathway.

8. The method of claim 1, wherein the anonymized patient health record data continually improve as new data becomes available.

9. The method of claim 1, wherein the classified anonymized patient health record data includes an operating point that balances measurements of specificity and sensitivity in order to effectively treat as many patients as possible.

10. The method of claim 1, wherein appropriate timing of the administration of the drug treatment reduces drug toxicity.

11. The method of claim 1, wherein the drug treatment is administration of resatorvid, eritoran, CytoFab, trigriluzole, or a 5-HT4 agonist.

12. A method of using a machine learning algorithm for administering a drug treatment to a candidate patient, comprising:
acquiring anonymized patient health record data from a plurality of patients;
comparing acquired patient data to the acquired anonymized patient health record data; and
administering the drug treatment to the candidate patient based on whether or not the timing of the drug treatment is efficacious in treating a specific manifestation of the candidate patient's condition as evidenced by the anonymized patient health record data.

13. The method of claim 12, wherein the anonymized patient health record data includes (i) patient demographics, (ii) measurements of vital signs, (iii) physiological monitor data, (iv) the ward in which the patient is staying, (v) diagnosis and treatment information, (vi) lab test results, (vii) medication data, (viii) patient outcome information, (ix) clinical notes, and/or (x) patient medical history.

14. The method of claim 12, wherein the anonymized patient health record data reflects the nature of the patient population served by the hospital or clinic in terms of patient demographics, rates of disease incidence, and/or treatment practices.

15. The method of claim 12, wherein the anonymized patient health record data is sourced from a database of the plurality of patients, a database of one or more care centers and patient populations, or from a database of multiple care centers and patient populations.

16. The method of claim 12, wherein the anonymized patient health record data is collected at a standard interval.

17. The method of claim 12, wherein the anonymized patient health record data includes at least one gold standard patient data that identifies that patient is progressing through a disease pathway for which the drug is expected to be effective.

18. The method of claim 17, wherein the at least one gold standard patient data includes a specific progression through the disease pathway.

19. The method of claim 12, wherein the anonymized patient health record data continually improve as new data becomes available.

20. The method of claim 12, wherein the classified anonymized patient health record data includes an operating point that balances measurements of specificity and sensitivity in order to effectively treat as many patients as possible.

21. The method of claim 12, wherein appropriate timing of the administration of the drug treatment reduces drug toxicity.

22. The method of claim 12, wherein the drug treatment is administration of resatorvid, eritoran, CytoFab, trigriluzole, or a 5-HT4 agonist.
